(19) United States
(12) Patent Application Publication
(10) Pub. No.: US 2002/0013362 A1
(43) Pub. Date: Jan. 31, 2002

(54) Paclitaxel treatment regimen for metastatic melanoma

(76) Inventor: Larry Helson, Quakertown, PA (US)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 09/837,682

(22) Filed: Apr. 18, 2001

Related U.S. Application Data
(60) Provisional application No. 60/198,055, filed on Apr. 18, 2000.

(51) Int. Cl. A61K 31/337

(52) U.S. Cl. 514/449

(57) ABSTRACT

The invention relates generally to a method of using paclitaxel in a specific Q4D×3 schedule and dosage for patients with malignant melanoma. The low risk-benefit ratio of this specific schedule provides a method for therapeutic use for all stages of severity of disease ranging from local resectable tumor to diagnosed widespread malignant melanoma.

### FIELD OF THE INVENTION

The present invention broadly concerns a treatment regimen for patients with local or disseminated malignant melanoma. More specifically, the present invention relates to a paclitaxel treatment schedule and dosages which are different from the standard drug schedule of once every 21 days.

### BACKGROUND OF THE INVENTION

Melanoma was once a rarity in oncological management. An exponential increase in incidence during the past 25 years has made it an important focus for treatment-related research. In 1995 there were an estimated 34,000 new cases and 7,000 deaths. (Wingo, Pa. 1995.) Treatment options and the effectiveness of systemic chemotherapy for advanced melanoma are limited, rendering it a fatal disease. Most agents and combinations of agents are no better than the single agent dacarbazine. (Legha 1990.) The results of scheduling for dacarbazine are quite variable depending upon the number of sequential days of treatment. Evidence is available that suggests that longer treatment schedules are more effective than shorter in producing overall response rates. (Nathanson, L. 1971; McClay E. F. and McClay, M. E., 1996.) These observations may be reflecting the pharmacodynamic characteristics of dacarbazine, particularly its dwell time in the cell and the duration of its biological effects. Based on this same premise, the pharmacodynamics of any drug considered for treatment of this disease must be taken into consideration, and in effect dictates the strategy for its schedule of administration.

Paclitaxel is a natural product extracted from the bark of Pacific yew trees, Taxus brevifolia. It has been shown to have excellent antitumor activity in in vivo animal models. Paclitaxel is a cell cycle specific agent which has as its primary intracellular target the beta subunit of tubulin, and when bound to it promotes and stabilizes the polymerized microtubular state. This causes metaphase arrest which in turn induces apoptotic cell death. Paclitaxel has been approved for the treatment of refractory advanced ovarian cancer and breast cancer; and studies involving other cancers have shown promising results. The results of paclitaxel clinical studies are reviewed by numerous authors (Hajek R. et al., 1996; Bedikian A. Y. et al., 1995; Spencer and Faulds, 1994; and Rowinsky and Donehower, 1991), and also in the references cited therein.

Disseminated melanoma, however, is an example of a malignancy which has exhibited an apparent low response rate and constitutive drug resistance to paclitaxel. For example, using a 24-hour infusion of paclitaxel at 250 mg/M² once every three weeks, partial responses in 3/25 patients with melanoma were reported (Legha S. S., 1990). A majority of the melanoma patients could not tolerate greater than 200 mg/M² paclitaxel (Legha 1990). Another phase II study, using the same schedule in 28 patients, resulted in five objective responses (Einzig, A. I., 1988). Since December 1992, a single dose of 130 mg/m² every 21 days has been the accepted norm. Data generated in other laboratories and clinical trials have failed to demonstrate more effective schedules and dosages for the treatment of melanoma (McClay, E. F. and McClay, M. E., 1996). Therefore, a current need exists for alternative schedules and dosages for the treatment of melanoma.

### SUMMARY OF THE INVENTION

In one aspect, the present invention provides a method of administration of paclitaxel which differs from contemporary usage and has several valuable features such as increased dose intensity, relatively diminished side effects, and equal or greater antitumor efficacy when compared with the once every 21-day 3-24 hour infusion schedule. The invention requires that the drug be given three times every 21 days at 3, 4, 5, or 6-day intervals. Each 21-day cycle is defined as a “course”. Specifically, the present invention concerns the preferred scheduling of paclitaxel chemotherapy at four-day intervals with the administration of paclitaxel dosages of about 60-175 mg/M²/day. Each course is to be repeated a minimum of three times in sequence over a 63-day period. In the absence of untoward toxicity, the treatments may be continued until the cancerous growth is stabilized, reduced, or destroyed. In a preferred embodiment, the application of a course of intravenous paclitaxel as a 90 mg/M² dose in a Q4D×3 schedule every 21 days for three or more courses for patients with malignant melanoma is a novel and clinically effective strategy differing from all contemporary standard regimens. Standard dosing regimens for paclitaxel are found, for example, in the product guidelines for the administration of TAXOL produced by Bristol-Meyers Squibb. Further, this invention is to be applied to all stages of disease ranging from the broadest use for patients with metastatic malignant melanoma who are chemotherapy naive, to its use as an adjuvant prophylactic measure for patients who have presumably locally resected disease. The technical design permits combination of the described treatment schedule with radiation therapy, other chemotherapy, vaccines, and biological therapeutic agents as well. Agents used in melanoma therapy include, but are not limited to, interferon alpha, cisplatin, carboplatin, carmustine, tamoxifen, and vinblastin. Even though most combinations are no better than the single agent dacarbazine, the addition of cytotoxic agents, biologic agents, and mitotic inhibitors such as paclitaxel has offered some promise. (McClay, E. F. and McClay, M. E., 1996.) The most preferred patient to apply this drug and schedule to is one with malignant melanoma which has been resected, and where the patient is at high risk for recurrence. In this setting the Q4 day (Q4D) schedule with its known efficacy and tolerability can be applied for 6 or more courses. Also preferred are malignant melanoma patients who are chemotherapy naive and in whom the application of paclitaxel in the Q4D schedule is given to control or eradicate disseminated, unresectable disease. Patients who are heavily pretreated with any chemotherapy may also benefit from this schedule/dosage strategy in terms of disease and symptomatic palliation. Improved clinical responses may follow the application of paclitaxel in combination with standard chemotherapeutic agents, cytokines, and radiation. The Q4D schedule lends itself to this strategy because of its ease of administration, limited toxicity, and fractionated scheduling.

In another embodiment, the tolerability of paclitaxel may differ according to the status of the patient and previous chemotherapeutic treatments, hence the dose intensity/course may range from 180-525 mg/M², and preferably from 270-330 mg/M². This is clearly demonstrated in acurrent melanoma salvage study being conducted at M D Anderson Cancer Center, Houston, Tex., U.S.A. The specific disease state as well as the previous chemotherapy can contribute to the degree of paclitaxel induced adverse events. Previous therapies and extensive disease also contribute to a variety of debilitated clinical statuses. The adverse effects of paclitaxel are generally minimal using the Q4D schedule at dose intensities greater than the maximally tolerated dosage possible with the once every 21 -day schedule. However, in very heavily pretreated patients the observed adverse effects can be serious. The most satisfactory representation of adverse effects from paclitaxel can be observed in chemotherapy naive, locally resected diseased patients where previous drug induced damage is negligible. Based upon published data from solid tumor and leukemic patients, 21-day dose intensities of 405 mg/M² are possible.

It is therefore an object of this invention to provide alternative schedules and dosages for using paclitaxel to treat melanoma.

### DETAILED DESCRIPTION OF THE PREFERRED EMBODIMENTS

By devising alternative schedules and dosages, we have discovered that more effective paclitaxel treatment regimens for melanoma do exist. Data from the NaPro BioTherapeutics laboratory utilizing in vitro human tumor cell lines and in vivo experiments with human heterotransplanted tumors in nude mice demonstrated that the therapeutic index and anticancer effects of paclitaxel could be improved. It was discovered that a fractionated schedule and a briefer infusion time is a clinically more effective strategy. Because of the smaller fractionated dose, paclitaxel can be given over a 45-80 minute infusion time. An added benefit to this regimen includes a reduction in myelosupression. To this end, we devised the Q4 day (Q4D)×3 every 21-day treatment schedule which has the added value of increasing the dose intensity during each 21-day treatment period beyond that of the once every 21-day schedule (Ainsworth S. K., and Helson, L., U.S. Pat. No. 5,696,153 issued Dec. 9, 1997). The safety of this Q4D paclitaxel schedule in adult leukemia (Seiter et al 1995), solid tumors (Helson et al. 1994, 1995), and pediatric solid tumor patients (Donfrancesco, A. 1995) has been published. During these trials an anecdotal twelve-month partial response in a patient with a recurrent large metastatic melanoma was observed. Currently, a phase II trial of the paclitaxel Q4D schedule at 90 mg/M²/dose in advanced heavily pretreated melanoma patients is being conducted at M D Anderson Hospital: The 21-day dose intensity in this schedule is 270 mg/M². Interim analysis reveals objective responses of greater than 50% tumor reduction and 25-40% reductions in hepatic tumor masses sustained over one month in an additional two patients of the first 21 patients accrued and treated. Associated toxicity was significantly less than the toxicity associated with lesser dose intensities of 200 mg/M² which are observed with the 200 mg/M²,Q 21-day schedule. Because of the apparent improved efficacy of this schedule, the reduced toxicity associated with brief infusions, and the greater dose intensity permitted with its application, this invention is a novel and important advance in the treatment of melanoma and furthers the understanding of the application of paclitaxel in the clinical setting. Because of the reduced associated toxicity, this schedule lends itself to paclitaxel associations with other anticancer modalities such as radiation, cytokines, and chemotherapy. Cytokines and therapeutic agents which may be used include, for example, platinum compounds (e.g., spiroplatin, cisplatin, and carboplatin), thalidomide, methotrexate, adriamycin, mitomycin, ansamitocin, bleomycin, cytosine arabinoside, arabinosyl adenine, mercaptopolylysine, vincristine, busulfan, chlorambucil, melphalan (e.g., PAM, L-PAM or phenylalanine mustard), mercaptopurine, mitotane, carmustine, procarbazine hydrochloride dactinomycin (actinomycin D), daunorubicin hydrochloride, doxorubicin hydrochloride, taxol, mitomycin, plicamycin (mithramycin), aminoglutethimide, estramustine phosphate sodium, flutamide, leuprolide acetate, megestrol acetate, tamoxifen citrate, testolactone, trilostane, amsacrine (m-AMSA), asparaginase (L-asparaginase) Erwina asparaginase, etoposide (VP-16), interferon α-2a, interferon α-2b, teniposide (VM-26), vinblastine sulfate (VLB), vincristine sulfate, bleomycin, bleomycin sulfate, methotrexate, adriamycin, and arabinosyl; biological response modifiers such as muramyldipeptide, muramyltripeptide, microbial cell wall components, lymphokines (e.g., bacterial endotoxin such as lipopolysaccharide, macrophage activation factor), sub-units of bacteria (such as Mycobacteria, Corynebacteria), the synthetic dipeptide N-acetyl-muramyl-L-alanyl-D-isoglutamine; hormones such as growth hormone, melanocyte stimulating hormone, estradiol, beclomethasone dipropionate, betamethasone, betamethasone acetate and betamethasone sodium phosphate, vetamethasone disodium phosphate, vetamethasone sodium phosphate, cortisone acetate, dexamethasone, dexamethasone acetate, dexamethasone sodium phosphate, flunisolide, hydrocortisone, hydrocortisone acetate, hydrocortisone cypionate, hydrocortisone sodium phosphate, hydrocortisone sodium succinate, methylprednisolone, methylprednisolone acetate, methylprednisolone sodium succinate, paramethasone acetate, prednisolone, prednisolone acetate, prednisolone sodium phosphate, prednisolone tebutate, prednisone, triamcinolone, triamcinolone acetonide, triamcinolone diacetate, triamcinolone hexacetonide and fludrocortisone acetate; immunoadjuvants and cytokines may also be employed, including interleukins (e.g., IL-1, IL-2, IL-3, IL-6), leukemia inhibitory factor, interferons, such as INF-α, TGF-beta, erythropoietin, TNF, lymphotoxin, and thrombopoietin; thalidomide and/or other agents to inhibit the production and/or action of cytokine/growth factors such as M-α, IGF-1, EGF, TGF α, IL-1β, MGSA and bFGF in different cell types involved in tumor growth and/or angiogenesis of melanoma.

Radiation therapy can be administered to the mammal according to protocols commonly employed in the art and known to the skilled artisan. Such therapy may include cesium, iridium, iodine, or cobalt radiation. The radiation therapy may be whole body irradiation, or may be directed locally to a specific site or tissue in or on the body, such as the lung, bladder, or prostate. Typically, radiation therapy is administered in pulses over a period of time from about 1 to about 2 weeks. The radiation therapy may, however, be administered over longer periods of time. For instance, radiation therapy may be administered to mammals having head and neck cancer for about 6 to about 7 weeks. Optionally, the radiation therapy may be administered as a single dose or as multiple, sequential doses.

As described herein, a range of dosages (60-175 mg/M² and preferably 90-120 mg/M²) of paclitaxel and alternative fractionated schedules (Q3-6 days) may be applied depending upon the clinical estimate of the patient's physiologic fragility consequent to extensive disease status and/or damage from previous treatment(s). In the following human studies, all patients had metastatic disease and were previously treated with one or more standard and experimental therapies. Hence, they presented with a variety of physiological statuses, and the adverse events following identical paclitaxel dosage and schedules varied according to the patient's condition at entry to the study. While the examples present the administration of paclitaxel in a specific fractionated schedule, Q4D×3 every 21 days, and dosage of 90 mg/M² in patients with malignant melanoma who have been previously treated and failed other therapies, it should be appreciated that a range of dosages (60-175 mg/M², and preferably 90-120 mg/M²) of paclitaxel and alternative fractionated schedules (Q3-6 days) may be applied depending upon the clinical estimate of the patients physiologic fragility consequent to extensive disease status and/or damage from previous treatment(s).

### EXAMPLE 1

In the current M D Anderson Trial, 17 patients with melanoma were given paclitaxel at 90 mg/M² Q4 days×3 (270 mg/M² q 21 days), and the following toxicities were noted: Grade 4 toxicity—7 times in 3 patients: BUN increase (1 patient, with greater than 10 times normal values), granulocytopenia (4 patients, less than 0.5×10³/mm³), and leukopenia (2 patients, less than 1.0×10³/mm³). Grade 3 toxicity—21 times in 11 patients: the observed toxicities included increased alkaline phosphatase levels (2 patients, between 5.1-20×N), anemia (2 patients, between 6.5-7.9 grams/100 ml blood), diarrhea (2 patients, over 7-9 stools/day), edema (1 patient, >20% weight gain), fatigue (4 patients, objective weakness with impairment of function), granulocytopenia (6 patients between 0.5-0.9×10³/mm³), abdominal pain (3 patients, sufficient to interfere with function), and sensory changes (1 patient, objective sensory loss or paresthesias interfering with function).

### EXAMPLE 2

Responses obtained in previously treated patients with metastatic melanoma. The patients received paclitaxel at 90 mg/M² Q4D×2-3 courses. It should be noted that patients with choroidal melanoma are notoriously resistant to any known type of chemotherapy and any responses with single drug chemotherapy are noteworthy (Am. J. Ophthalmol 1996).

Patient #1. 46-year-old male with a subungal primary and liver metastases failed interferon and bio-chemotherapy with interleukin-2, cisplatin, velban, and Ditrioimadazole carbamoxamide (DTIC). After two courses of paclitaxel at 270 mg/M²/course the patient exhibited a 41% decrease in liver metastases volume. After another two courses, tumor volume decreased 83%.

Patient #2. 51-year-old male with a left calf primary diagnosed in 1995; in 1998 he developed metastases to femoral and inguinal nodes and lung. Patient failed cisplatin, velban, DTIC, and melanoma vaccine. Patient exhibited a 43% decrease in lung metastases and a similar decrease in volume of inguinal nodes. Tumor control was maintained through eight courses at 270 mg/M²/course. The major side effect was paresthesia.

Patient #3. A 49-year-old male with a scapular primary exhibited a 39% decrease in tumor volume after 4 courses at 270 mg/M²/course.

Patient #4. A 44-year-old female with a right calf primary exhibited a 15% decrease in tumor volume after 4 courses at 270 mg/M²/course.

Patient #5. A 56-year-old male with a right heel mass exhibited a 12% decrease in tumor volume after 2 courses at 270 Mg/M²/course.

Patient #6. A 79-year-old male exhibited stable disease during 4 courses of paclitaxel at 270 mg/M²/course.

Patient #7. A 37-year-old female with a ciliochoroidal primary tumor and no previous chemotherapy exhibited stable disease after 3 courses of paclitaxel at 270 mg/M²/course.

Patient #8. A 56-year-old male previously treated with chemotherapy, and with a tumor of the dorsum of the right foot exhibited stable disease during two courses of paclitaxel at 270 mg/M²/course.

Patient #9. A 76-year-old female with no prior chemotherapy and a primary non-choroidal melanoma exhibited stable disease during 6 courses of paclitaxel at 270 mg/M²/course.

According to the present invention therefore, a method for administration of paclitaxel is provided for patients suffering from malignant melanoma. This method comprises a treatment strategy of infusing an amount of paclitaxel of 60-175 mg/M², and preferably 90-120 mg/M², over a period of 60 minutes at least 3 times at 3-6 day intervals over a 21-day period. The preferred schedule is every 4 days, and the preferred dosage is 90 mg/M²/dose with at least three 21-day courses. Additional 21-day courses are recommended in the presence of partial clinical responses and tolerable toxicities. The minimum amount of paclitaxel administered over a 63-day period is 810 mg/M². Thus, according to the present invention, dosages surpassing the maximum tolerated dose intensity of 525 mg/M² under previous regimens (three 175 mg/M² doses given every 21 days) exhibit increased efficacy and reduced levels of toxicity as compared to previous regimens.

### References

Am. J. Ophthalmol,122:106-108 (1996).

Bedikian 1999, unpublished data from M D Anderson Study of Q4D paclitaxel in Malignant Melanoma.

Bedikian, A. Y. et al., “Phase II trial of docetaxel in patients with advanced cutaneous malignant melanoma previously untreated with chemotherapy,” J. Clin. Oncol., 13(12):2895-2899.

Donfrancesco, A., Deb, G., Sio, L., Habettswallner, D., Helson, L. “Phase I trial of a Q4D Taxol regimen in pediatric patients with recurrent solid tumors.” American Society of Clinical Oncology, May 1995.

Einzig, A. I., Trump, D. L., Sasloff, J. et al. “Phase II pilot study of taxol in patients with malignant melanoma.” Proc. Am. Soc. Clin. Oncol.7:249,1988.

Hajek, R. et al., “Paclitaxel (Taxol): a review of its antitumor activity in clinical studies Minireview,” Neoplasma, 43(3): 141-154, 1996.

Helson, L., Puccio, M. Ostrow, S., Mittleman, A., Ahmed, T. “Phase I Taxol; Fractionated Q4D brief infusional schedule in advanced solid tumors.” American Society of Clinical Oncology May 1994.

Legha, S. S., Ring, S., Papadopoulos, N. et al. Cancer 11:2478-2481, 1990.

McClay, E. F., and McClay, M. E. “Systemic Chemotherapy for the treatment of Metastatic Melanoma.” Seminars in Oncology 23:744-753, 1996.

Nathanson, L., Wolter, L., Horton, J. et al. “Characteristics of prognosis and response to an imidazole carboxamide in malignant melanoma.” Clin. Pharmacol. Ther 12:955-962, 1971.

Rowinsky, K. and Donehower, R. C., “The Clinical Pharmacology and Use of Antimicrotubule Agents in Cancer Chemotherapeutics,” Pharmac. Ther., 52: 35-84, 1991.

Spencer, C. M. and Faulds, D., “Paclitaxel, A Review of its Pharmacodynamic and Pharmacokinetic Properties and Therapeutic Potential in the Treatment of Cancer,” Drugs, 48(5), 794-847, 1994.

Wingo, P. A., Tong, T., Bolder, S. “Cancer Statistics.” CA Cancer J. Clin.45:8-30, 1995.

U.S. Pat. No. 5,696,153 issued Dec. 9, 1997 to Ainsworth S. K., and Helson, L.

1. An article of manufacture comprising:
a container;
a pharmaceutically acceptable cytotoxic composition comprising paclitaxel disposed within said container; and
accompanying said container, instructions for administration of said paclitaxel composition to a patient suffering from melanoma, said instructions directing the administration of paclitaxel to the patient in a dosage which is in the range of from 60 mg/M²/day to 175 mg/M²/day three times during a twenty-one day course, at intervals selected from the group consisting of three, four, five, and six-day intervals.

2. An article of manufacture according to claim 1, wherein said instructions further comprise directing administration of paclitaxel over at least three, twenty-one day courses sequentially over a sixty-three day period.

3. An article of manufacture according to claim 1, wherein said instructions further comprise directing the administration of said daily paclitaxel doses over an infusion time which is in the range of from 45 minutes to 80 minutes.

4. An article of manufacture according to claim 1, wherein the interval of paclitaxel administration is three days.

5. An article of manufacture according to claim 1, wherein the interval of paclitaxel administration is four days.

6. An article of manufacture according to claim 1, wherein the interval of paclitaxel administration is five days.

7. An article of manufacture according to claim 1, wherein the interval of paclitaxel administration is six days.

8. An article of manufacture according to claim 1, wherein said paclitaxel dosage range is from 90 mg/M²/day to 120 mg/M²/day.

9. An article of manufacture according to claim 2, wherein said paclitaxel dosage range is from 90 mg/M²/day to 120 mg/M²/day.

10. An article of manufacture according to claim 3, wherein said paclitaxel dosage range is from 90 mg/M²/day to 120 mg/M²/day.

11. An article of manufacture according to claim 4, wherein said paclitaxel dosage range is from 90 mg/M²/day to 120 mg/M²/day.

12. An article of manufacture according to claim 5, wherein said paclitaxel dosage range is from 90 mg/M²/day to 120 mg/M²/day.

13. An article of manufacture according to claim 6, wherein said paclitaxel dosage range is from 90 mg/M²/day to 120 mg/M²/day.

14. An article of manufacture according to claim 7, wherein said paclitaxel dosage range is from 90 mg/M²/day to 120 mg/M²/day.

15. An article of manufacture according to claim 8, wherein said dosage is 90 mg/M²/day.

16. An article of manufacture according to claim 9, wherein said dosage is 90 mg/M²/day.

17. An article of manufacture according to claim 10, wherein said dosage is 90 mg/M²/day.

18. An article of manufacture according to claim 11, wherein said dosage is 90 mg/M²/day.

19. An article of manufacture according to claim 12, wherein said dosage is 90 mg/M²/day.

20. An article of manufacture according to claim 13, wherein said dosage is 90 mg/M²/day.

21. An article of manufacture according to claim 14, wherein said dosage is 90 mg/M²/day.

22. An article of manufacture according to claim 3, wherein said instructions direct an infusion time of 60 minutes.

23. An article of manufacture according to claim 8, wherein said instructions direct an infusion time of 60 minutes.

24. An article of manufacture according to claim 9, wherein said instructions direct an infusion time of 60 minutes.

25. An article of manufacture according to claim 10, wherein said instructions direct an infusion time of 60 minutes.

26. An article of manufacture according to claim 11, wherein said instructions direct an infusion time of 60 minutes.

27. An article of manufacture according to claim 12, wherein said instructions direct an infusion time of 60 minutes.

28. An article of manufacture according to claim 13, wherein said instructions direct an infusion time of 60 minutes.

29. An article of manufacture according to claim 14, wherein said instructions direct an infusion time of 60 minutes.

30. A method of administering paclitaxel to a patient with melanoma, comprising the steps of:
observing a patient having malignant melanoma; and
administering to said patient paclitaxel at a dose which is in the range of from 60 mg/M²/day to 175 mg/M²/day three times during a twenty-one day course, at intervals selected from the group consisting of three, four, five, and six-day intervals.

31. A method of administering paclitaxel according to claim 30, wherein said administration of paclitaxel is over at least three, twenty-one day courses sequentially over a sixty-three-day period.

32. A method of administering paclitaxel according to claim 30, wherein said administration of each of the daily paclitaxel doses is over an infusion time which is in the range of from 45 minutes to 80 minutes.

33. A method of administering paclitaxel according to claim 30, wherein the interval of paclitaxel administration is three days.

44. A method of administering paclitaxel according to claim 37, wherein said dosage is 90 mg/M²/day.

45. A method of administering paclitaxel according to claim 38, wherein said dosage is 90 mg/M²/day.

46. A method of administering paclitaxel according to claim 39, wherein said dosage is 90 mg/M²/day.

47. A method of administering paclitaxel according to claim 40, wherein said dosage is 90 mg/M²/day.

48. A method of administering paclitaxel according to claim 41, wherein said dosage is 90 mg/M²/day.

49. A method of administering paclitaxel according to claim 42, wherein said dosage is 90 mg/M²/day.

50. A method of administering paclitaxel according to claim 43, wherein said dosage is 90 mg/M²/day.

51. A method of administering paclitaxel according to claim 32, wherein the infusion time is 60 minutes.

52. A method of administering paclitaxel according to claim 37, wherein the infusion time is 60 minutes.

53. A method of administering paclitaxel according to claim 38, wherein the infusion time is 60 minutes.

54. A method of administering paclitaxel according to claim 39, wherein the infusion time is 60 minutes.

55. A method of administering paclitaxel according to claim 40, wherein the infusion time is 60 minutes.

56. A method of administering paclitaxel according to claim 41, wherein the infusion time is 60 minutes.

57. A method of administering paclitaxel according to claim 42, wherein the infusion time is 60 minutes.

58. A method of administering paclitaxel according to claim 43, wherein the infusion time is 60 minutes.
